(19) United States
(12) Patent Application Publication
(10) Pub. No.: US 2012/0089332 A1
(43) Pub. Date: Apr. 12, 2012

(54) System for Detecting Oil Spills and Method Thereof

(75) Inventor: Sungwook HONG, Seoul (KR)

(73) Assignee: KOREA METEOROLOGICAL ADMINISTRATION, Seoul (KR)

(21) Appl. No.: 13/269,180

(22) Filed: Oct. 7, 2011

(30) Foreign Application Priority Data
Oct. 8, 2010 (KR) 10-2010-0098204

(51) Int. Cl. G06F 19/00; G01N 33/26

(52) U.S. Cl. 702/2

(57) ABSTRACT

Disclosed are systems and methods of detecting oil spills on the sea surface at night. According to some embodiments, implementations herein involve detection of the polarized reflectivity and the refractive index of the water and the oil using the polarization properties of the electromagnetic waves based on satellite data to accurately and quantitatively detect the position of the oil band spread on the sea.

### CROSS-REFERENCE(S) TO RELATED APPLICATION

This application claims priority of Korean Patent Application No. 10-2010-0098204, filed on Oct. 8, 2010, in the Korean Intellectual Property Office, which is hereby incorporated by reference in its entirety.

### BACKGROUND OF THE DISCLOSURE

1. Field of the Disclosure

The present disclosure relates to a system for detecting oil spills on the sea at night using refractive index calculation based on satellite observation, and a method thereof.

2. Description of the Related Art

Oil spills on the sea cause environmental disaster near the waters, require a great human and physical effort for purification, and cause more economic damage to fisheries or tourism, etc. Although it is possible to detect the oil band using aircraft, only a satellite method can be used for observing the whole oil band at the same time and detecting a change process in the oil band. There are various methods, using visible, ultraviolet, infrared spectral bands, radar, laser, etc., in satellite remote sensing. These methods can detect the oil band by day only. Night detection using an infrared channel is not difficult to implement through the present technologies.

In recent years, the oil spill off the Taean Peninsula in South Korea and the continuous spill due to the explosion at a deep sea oilfield in the Gulf of Mexico near the United States have caused huge environmental disasters. The existing methods depending on satellite observation use various spectrums from ultraviolet to microwave bands, but have drawbacks in mainly detecting the oil spills in daytime only and a false signal.

### SUMMARY OF THE DISCLOSURE

An aspect of the present disclosure is directed to a method and a system for verifying how much oil band is spreading on the sea surface by detecting polarized reflectivity and a refractive index of water and oil using polarization properties of electromagnetic waves based on satellite data.

According to an embodiment of the present disclosure, the embodiment may obtain reflectivity by a ratio of radiance observed from a satellite to estimated sea surface temperature and calculate two reflectivities using polarization properties of electromagnetic waves according to surface properties. In this case, physical properties of water are different from those of oil and the reflective index values of the water and the oil are different from each other, thereby detecting oil spills on the surface.

The exemplary embodiments of the present disclosure may detect how much oil is spreading by obtaining a refractive index and reflectivity polarization component of an oil band exposed on the sea surface.

### BRIEF DESCRIPTION OF THE DRAWINGS

FIG. 1 is a diagram showing a configuration of a system according to an exemplary embodiment of the present disclosure.

FIG. 2 is a diagram showing a flow chart for detecting an oil spill position on the sea surface using the system according to the disclosure.

FIG. 3 is a diagram showing an example for radiance, cloud detection, sea surface temperature, and emission rate using the satellite infrared sensors and is a diagram showing results obtained by verifying the above-mentioned methods.

FIG. 4 is a diagram showing an example for oil detection, using a refractive index on the sea surface, using the satellite infrared sensors and is a diagram showing results obtained by verifying the above-mentioned method.

FIGS. 5-10 are diagrams showing an examplary implementation, in software, of a configuration of the oil detection system according to the exemplary embodiment of the present disclosure.

### REFERENCE NUMERALS

100: OBSERVATION SENSOR UNIT OF THE SATELLITE
200: NON-POLARIZATION REFLECTIVITY DETERMINING UNIT
300: REFRACTIVE INDEX OPERATION UNIT
400: OIL DETECTION ANALYSIS UNIT

### DETAILED DESCRIPTION OF EXEMPLARY EMBODIMENTS

Exemplary embodiments of the present disclosure will be described below in detail with reference to the accompanying drawings. Wherever possible, the same reference numerals will be used to refer to the same elements throughout the specification, and a duplicated description thereof will be omitted. It will be understood that although the terms “first”, “second”, etc. are used herein to describe various elements, these elements should not be limited by these terms. These terms are only used to distinguish one element from another element.

Aspects of the present disclosure may involve a detection method using a refractive index, i.e., a difference in material characteristic between water and oil to provide a method and a system for detecting oil spills using a satellite at night, which could not be solved by the existing methods.

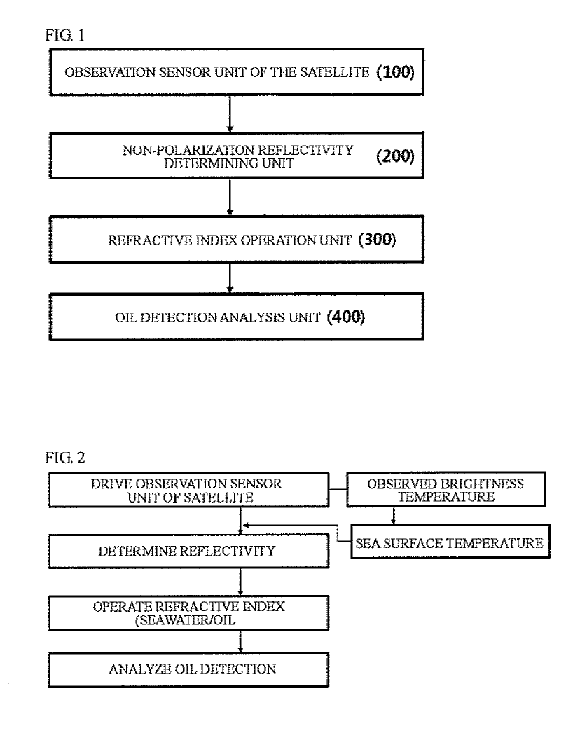

FIG. 2 shows a block diagram of a configuration of an oil detection system (hereinafter, referred to as ‘the present system’) detecting oil spilled on the sea surface according to the exemplary embodiment of the present disclosure.

Referring to FIG. 2, the present system includes a non-polarization reflectivity determining unit 200 determining vertical emission rate, horizontal emission rate or reflectivity for each polarization for sea water and oil of the sea surface region to which oil is spilled, using radiance measured by an observation sensor unit of the satellite, a refractive index operation unit 300 obtaining the refractive index of the sea water and the oil using the vertical reflectivity or the horizontal reflectivity determined by the non-polarization reflectivity determining unit, and an oil detection analysis unit analyzing the refractive index of the sea water and the oil and discriminating the position of the oil.

The observation sensor unit of the satellite 100 may use a near infrared channel of an infrared sensor mounted in the satellite, and the satellite uses MODIS (Moderate Resolution Imaging Spectroradiometer) data of Aqua, that is, the polar orbit satellite of the United States of America (USA). Using MODIS 11 μm channel as the observation channel is described by way of example.

The non-polarization reflectivity determining unit 200 obtains emission rate and vertical reflectivity or horizontal reflectivity for each polarization using radiance and the sea surface temperature measured by observation sensor unit including the infrared sensor of the satellite, wherein the reflectivity R(θ) and the vertical reflectivity R_{V} or the horizontal reflectivity R_{H} may be calculated according to Equations 1 and 2 stated below. Further, observed brightness temperature IB may use various satellite data; however, MODIS data of the polar orbit satellite called Aqua of USA are used herein. The MODIS data are used universally. The sea surface temperature Ts is difficult to directly observe over a vast region and therefore, the MODIS data are used.

$$R ( θ ) \approx 1 - \frac{{I}_{B} ( θ )}{B ( {T}_{S} )} \qquad \{ Equation 1 \}$$

(wherein θ is an observation angle of the satellite, R(θ) is the unpolarized surface reflectivity).

$${R}_{V} = {R}_{H}^{{sec}^{2} θ} \qquad \{ Equation 2 \}$$

(However, θ is the observation angle of the satellite and V and H represent vertical and horizontal polarization.)

That is, reflectivity for each polarization is obtained using the radiance and the sea surface temperature measured by the infrared sensor of the satellite, and the reflectivity for each such polarization component is represented differently for each substance. Therefore, the refractive index of the sea water and the oil are obtained using the reflectivity for each substance that is represented differently.

The refractive index is calculated by the refractive index operation unit 300 and may be calculated using the following Equation 3. The refractive index of the sea water and oil band may be operated according to {Equation 3} using the reflectivity provided by the non-polarization reflectivity determining unit 200.

$$n = \sqrt{\frac{{B}^{2} - {A}^{2} + {sin}^{2} θ + \sqrt{{( {A}^{2} + {B}^{2} - {sin}^{2} θ )}^{2} + 4 {A}^{2} {B}^{2}}}{2}} \qquad \{ Equation 3 \}$$

(However, A²=B²−2ab cos θ−cos²θ, B=(a−b)sin θ cot 2θ/[ab+(1−a²)cos²θ−1], coefficients a and b are a combination of reflectivities and are given like a=(R_{V}+1)/(R_{V}−1), b=(R_{H}+1)/(R_{H}−1))

That is, the refractive index is calculated using the above-described present system, and physical characteristics of the sea water and the oil band are distinctly analyzed to accurately detect the position of the oil spill. In other words, the reflectivity is obtained by a ratio of the radiance observed from the satellite to the estimated sea surface temperature, and two reflectivities are calculated using polarization properties of electromagnetic waves according to surface properties. Since the physical properties of the water are different from those of the oil, the refractive index values of the water and the oil are different from each other, thereby detecting the oil spilled on the sea surface. Thereby, we may detect how much oil is spreading by obtaining the refractive index and reflectivity polarization components of the oil band exposed on sea surface.

In addition, implementations of the present method may detect oil spread out over the sea at night using the infrared channel mounted in the satellite. As the advantages of the present method, the oil band may be detected by day and night, thereby accurately detecting and predicting the spreading of the oil band.

Method of detecting the oil using the above-described implementations may include determining vertical emission rate, horizontal emission rate or reflectivity for each polarization for the sea water and the oil of the oil spilled sea surface region, using radiance measured by an observation sensor unit of the satellite, and obtaining the refractive index of the sea water and the oil using the R_{V} or R_{H} obtained via such determining processes. Further, implementations herein may include comparing the refractive indexes of the operated sea water and oil to detect the oil spilled region. The determining processes may be performed via the non-polarization reflectivity determining unit, which may obtain the emission rate and the vertical reflectivity or the horizontal reflectivity for each polarization using radiance and the sea surface temperature measured by the observation sensor unit including the infrared sensor of the satellite, wherein the reflectivity R(θ) and vertical reflectivity R_{V} or horizontal reflectivity R_{H} may be calculated according to {Equation 1} and {Equation 2} as stated above.

Thereafter, the refractive indexes of the sea water and the oil band may be processed according to the above steps {Equation 3} using the reflectivity provided from the determining by the refractive index operation unit, such that a spreading degree of the oil band may be detected based on the difference in the refractive indexes between two substances.

Implementations of the present disclosure are applicable to a variety of industries such as weather, climate, environment, disaster prevention, etc. Here, for example, the present systems and methods involve innovative aspects for detecting the refractive index for the oil band on the sea at night to the known position of the spilled oil, thereby providing very useful information to warn of or forecast the oil spill.

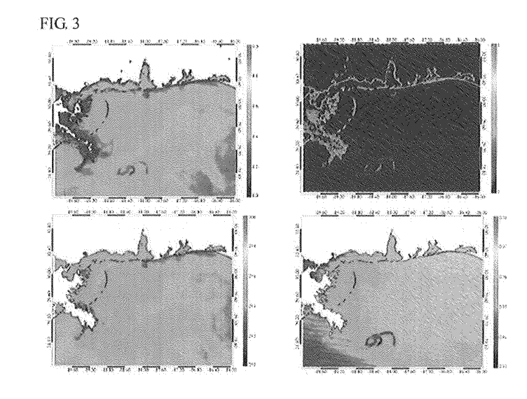

FIG. 3 is an example for radiance, cloud detection, sea surface temperature, and emission rate using the satellite infrared sensors, and shows the results obtained by verifying the method presented above.

Specifically, FIG. 3 shows radiance, cloud information, sea surface temperature, and emissivity (=1-reflectivity) observed using the Aqua, that is, the polar orbit satellite of the USA, and shows the results obtained by verifying the satellite data and the above-described method.

The actual example of the oil spills may be an example of the oil spill off the coast of the Gulf of Mexico on Apr. 29, 2010. Although the oil band is shown in a swirl shape, the current Aqua satellite data classify the oil band by the cloud. Although the oil band in the swirl shape is shown when using the emission rate, when the oil band is present at the blue portion of the lower left plane, it is impossible to classify the oil band. This relies on the attention angle for the satellite observation.

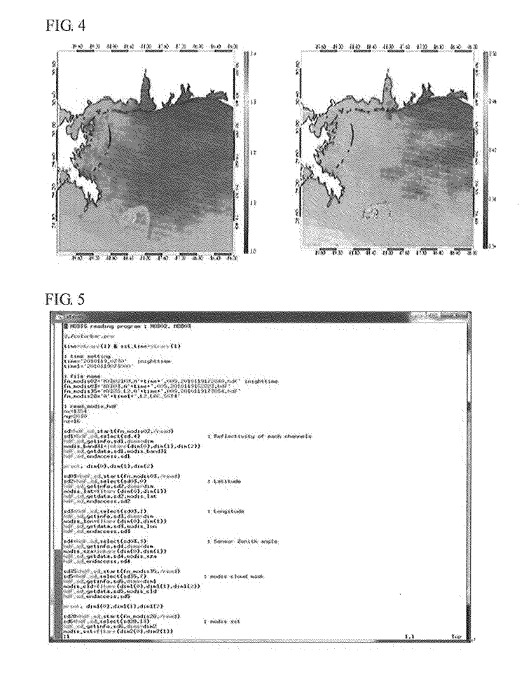

FIG. 4 is an example for oil detection, using refractive index on the sea surface, using the satellite infrared sensors, and shows the results verifying obtained by the method presented above.

Specifically, FIG. 4 shows the real part and the imaginary part of the refractive index calculated using the same satellite data of the same date as FIG. 3, respectively. The two components exhibit the oil band characteristic in the swirl shape as shown in FIG. 1. However, if the oil band is positioned at the bottom left due to the attention angle, it is difficult to classify the oil band by the real part only but the characteristic is clearly exhibited when using the imaginary part. Therefore, very useful information to detect whether the oil band is present may be additionally provided by providing two data that are not provided in the related art.

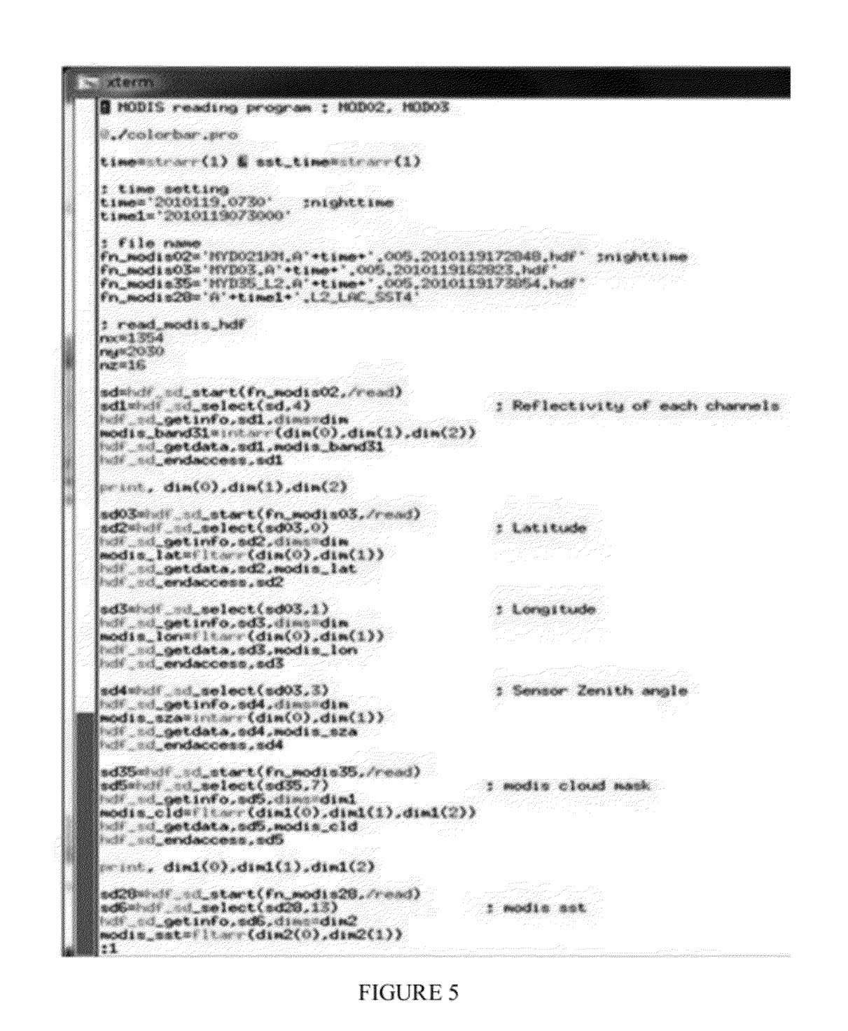

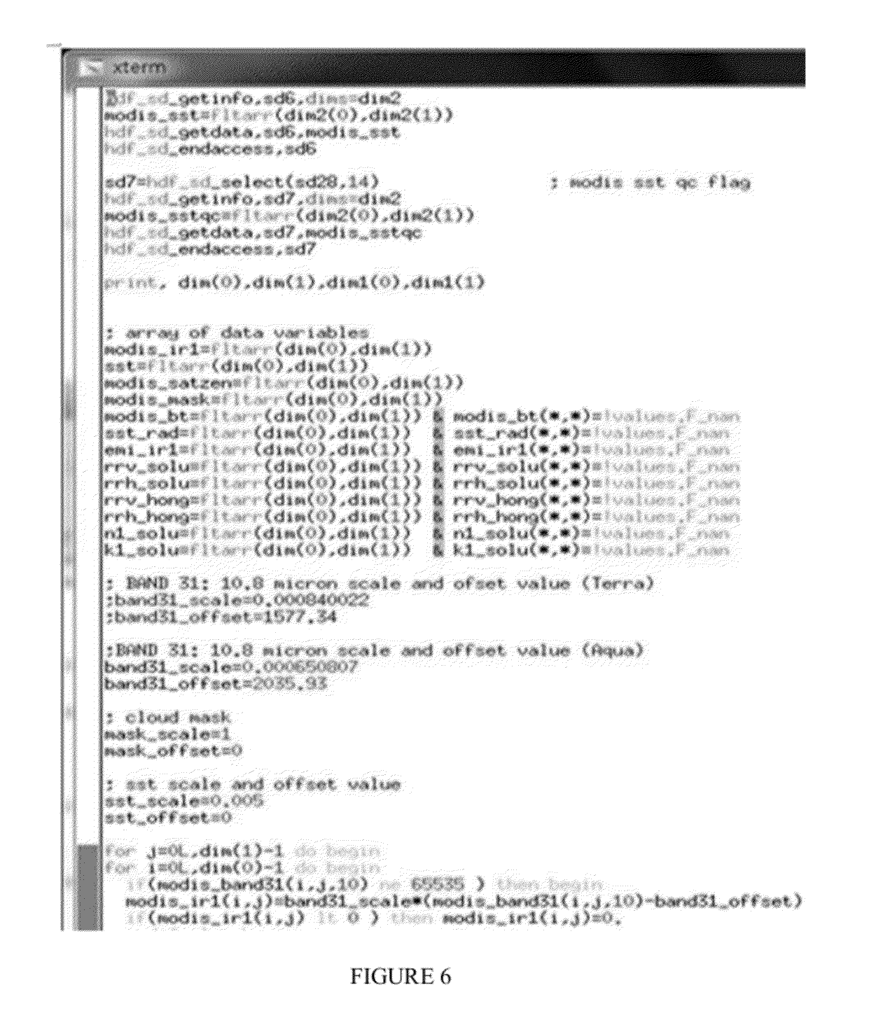

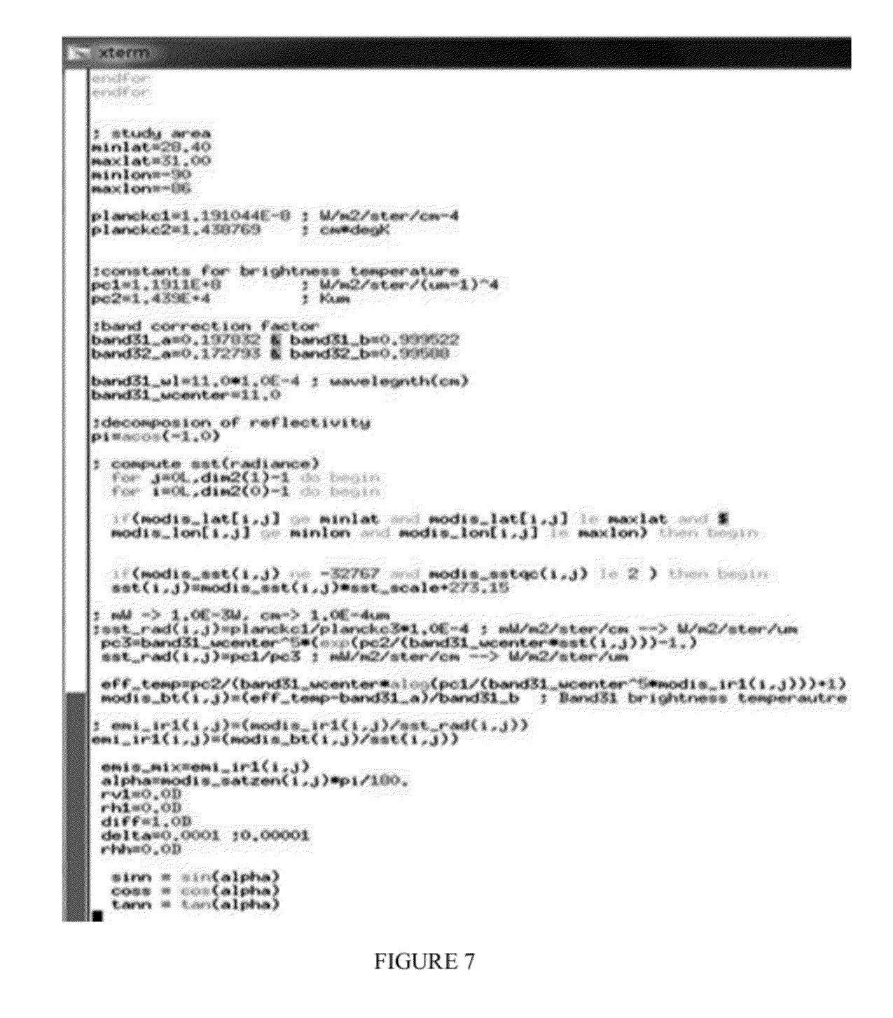

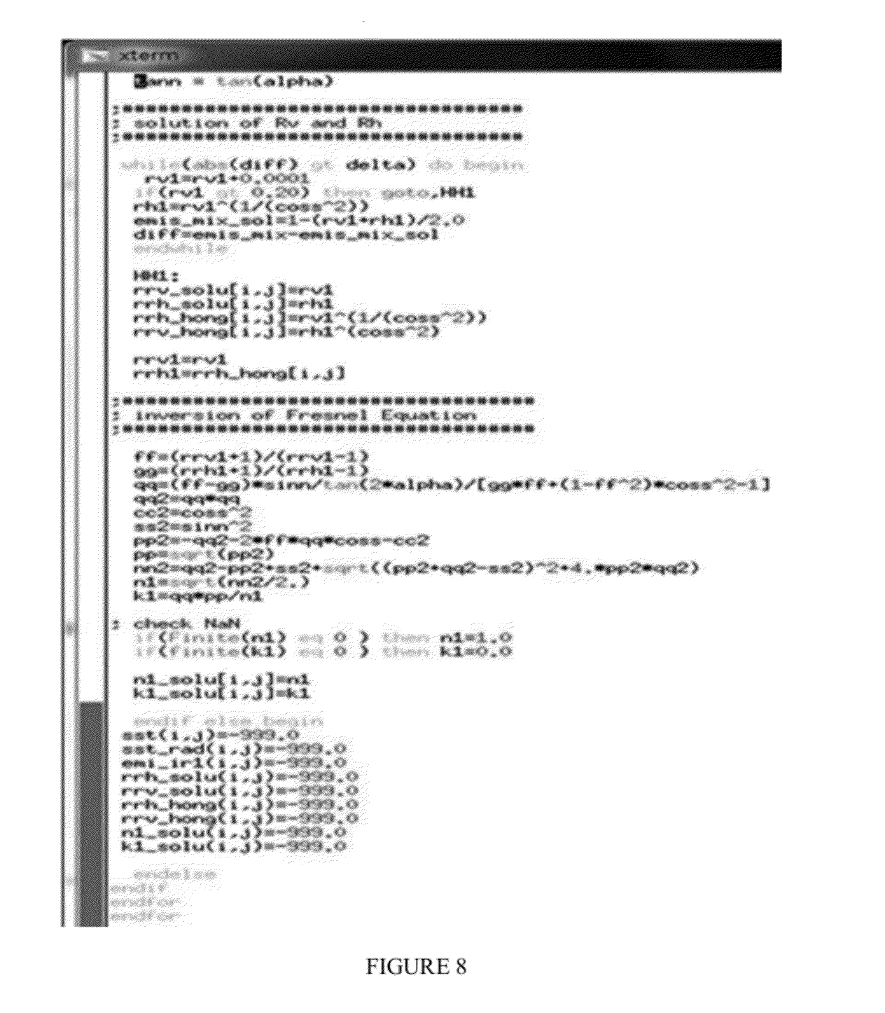

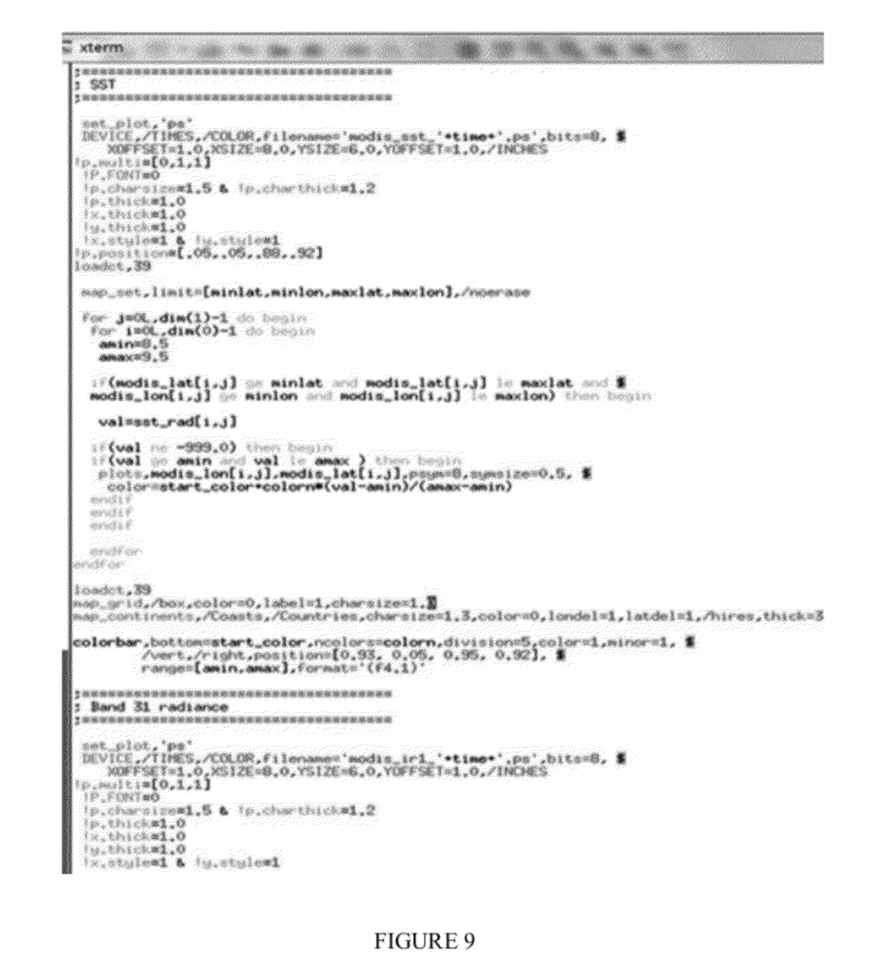

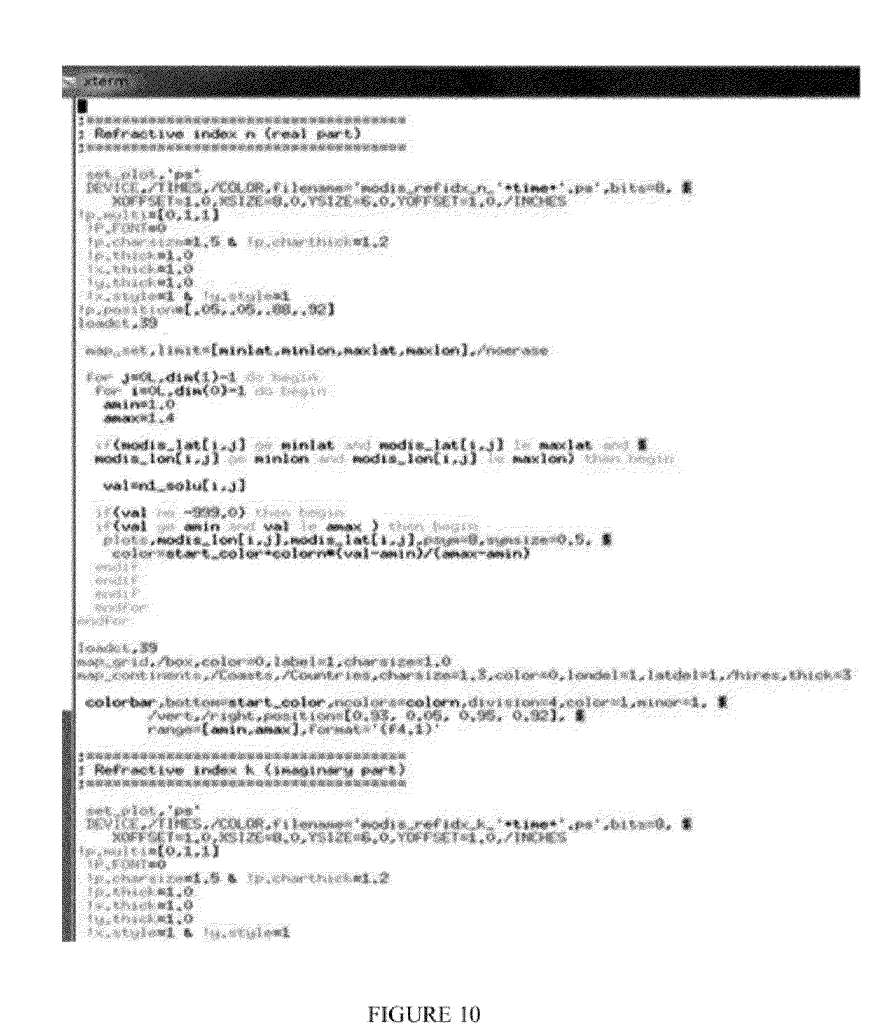

FIGS. 5-10 show an exemplary implementation, in software, of a configuration of the oil detection system according to the exemplary embodiment of the present disclosure. As such, the system and method according to the exemplary embodiment of the present disclosure may be configured in software and therefore, may be manufactured in the form of computer-readable recording medium including programs to execute the system and the method.

As set forth above, the exemplary embodiments of the present disclosure can detect the polarized reflectivity and the refractive index of the water and the oil using the polarization properties of the electromagnetic waves based on the satellite data to accurately and quantitatively detect the position of the oil band spread on the sea.

In particular, the exemplary embodiments of the present disclosure can know the refractive indexes based on the satellite observation and therefore, detect the oil band distinguished from the sea water using the difference in the refractive index of the water and the oil at night. Therefore, the exemplary embodiments of the present disclosure can be very usefully used for environmental problems such as oil spills, and in particular, can easily confirm the spread region and be applied to predict the spread course to give advance warning to the area in which disaster may occur, thereby reducing economic, human and material damages.

While the disclosure has been shown and described with reference to exemplary embodiments thereof, it will be understood by those skilled in the art that various changes in form and details may be made therein without departing from the spirit and scope of the disclosure as defined by the appended claims. Therefore, the scope of the disclosure is defined not by the detailed description of the disclosure but by the appended claims, and all differences within the scope will be construed as being included in the present disclosure.

1. A system for detecting oil spills on the sea surface, comprising:
a non-polarization reflectivity determining unit that determines reflectivity for each polarization for sea water and oil of a sea surface region on which oil is spilled, using radiance measured by an observation sensor unit of a satellite;
a refractive index operation unit that processes refractive indexes of sea water and oil using vertical reflectivity or horizontal reflectivity determined by the non-polarization reflectivity determining unit; and
an oil detection analysis unit that analyzes the refractive indexes of the sea water and the oil and discriminating a position of the oil.

2. The system of claim 1, wherein the non-polarization reflectivity determining unit obtains emission rate and vertical reflectivity or horizontal reflectivity for each polarization using the radiance and the sea surface temperature measured by the observation sensor unit including the infrared sensor of the satellite, where the reflectivity R(θ) and vertical reflectivity R_{V} or horizontal reflectivity R_{H} are calculated according to Equations 1 and 2 stated below,
$$R ( θ ) \approx 1 - \frac{{I}_{B} ( θ )}{B ( {T}_{S} )} \qquad \{ Equation 1 \}$$
where θ is an observation angle of the satellite
$${R}_{V} = {R}_{H}^{{sec}^{2} θ} \qquad \{ Equation 2 \}$$
where θ is the observation angle of the satellite and V and H represent vertical and horizontal polarizations, respectively.

3. The system of claim 2, wherein the refractive index operation unit operates the refractive indexes of the sea water and the oil band according to {Equation 3} using the reflectivity provided by the non-polarization reflectivity determining unit,
$$n = \sqrt{\frac{{B}^{2} - {A}^{2} + {sin}^{2} θ + \sqrt{{( {A}^{2} + {B}^{2} - {sin}^{2} θ )}^{2} + 4 {A}^{2} {B}^{2}}}{2}} \qquad \{ Equation 3 \}$$
where A²=B²−2ab cos θ−cos²θ, B=(a−b)sin θ cot 2θ/[ab+(1−a²) cos²θ−1], coefficients a and b are a combination of reflectivities, wherein a=(R_{V}+1)/(R_{V}−1), and b=(R_{H}+1)/(R_{H}−1).

4. A method for detecting oil spills on a sea surface, comprising:
determining vertical emission rate, horizontal emission rate or reflectivity for each polarization for sea water and oil of a sea surface region on which oil is spilled, using radiance measured by a sensor mounted on a satellite, and
obtaining the refractive index of the sea water and the oil using vertical reflectivity or horizontal reflectivity information determined at the determining step.

5. The method of claim 4, further comprising using the refractive indexes of the operated sea water and the oil to detect the region on which the oil is spilled.

6. The method of claim 4, wherein the determining obtains emission rate and vertical reflectivity or horizontal reflectivity for each polarization using the radiance and the sea surface temperature measured by the observation sensor unit including the infrared sensor of the satellite, where the reflectivity R(θ) and vertical reflectivity R_{V} or horizontal reflectivity R_{H} are calculated according to Equations 1 and 2 stated below,
$$R ( θ ) \approx 1 - \frac{{I}_{B} ( θ )}{B ( {T}_{S} )} \qquad \{ Equation 1 \}$$
where θ is an observation angle of the satellite
$${R}_{V} = {R}_{H}^{{sec}^{2} θ} \qquad \{ Equation 2 \}$$
where θ is the observation angle of the satellite and V and H represent vertical and horizontal polarizations, respectively.

7. The method of claim 6, wherein the obtaining step processes the refractive indexes of the sea water and the oil band according to {Equation 3} using the reflectivity provided by the determining
$$n = \sqrt{\frac{{B}^{2} - {A}^{2} + {sin}^{2} θ + \sqrt{{( {A}^{2} + {B}^{2} - {sin}^{2} θ )}^{2} + 4 {A}^{2} {B}^{2}}}{2}} \qquad \{ Equation 3 \}$$
where A²=B²−2ab cos θ−cos²θ, B=(a−b)sin θ cot 2θ/[ab+(1−a²) cos²θ−1], coefficients a and b are a combination of reflectivities, wherein a=(R_{V}+1)/(R_{V}−1), and b=(R_{H}+1)/(R_{H}−1).

8. (canceled)

9. The method of claim 5, wherein the determining obtains emission rate and vertical reflectivity or horizontal reflectivity for each polarization using the radiance and the sea surface temperature measured by the observation sensor unit including the infrared sensor of the satellite, where the reflectivity R(θ) and vertical reflectivity R_{V} or horizontal reflectivity R_{H} are calculated according to Equations 1 and 2 stated below,
$$R ( θ ) \approx 1 - \frac{{I}_{B} ( θ )}{B ( {T}_{S} )} \qquad \{ Equation 1 \}$$
where θ is an observation angle of the satellite
$${R}_{V} = {R}_{H}^{{sec}^{2} θ} \qquad \{ Equation 2 \}$$
where θ is the observation angle of the satellite and V and H represent vertical and horizontal polarizations, respectively.

10. The method of claim 9, wherein the obtaining step processes the refractive indexes of the sea water and the oil band according to {Equation 3} using the reflectivity provided by the determining
$$n = \sqrt{\frac{{B}^{2} - {A}^{2} + {sin}^{2} θ + \sqrt{{( {A}^{2} + {B}^{2} - {sin}^{2} θ )}^{2} + 4 {A}^{2} {B}^{2}}}{2}} \qquad \{ Equation 3 \}$$
where A²=B²−2ab cos θ−cos²θ, B=(a−b)sin θ cot 2θ/[ab+(1−a²) cos²θ−1], coefficients a and b are a combination of reflectivities, wherein a=(R_{V}+1)/(R_{V}−1), and b=(R_{H}+1)/(R_{H}−1).

11. At least one computer-readable medium comprising computer-readable instruction operable to cause one or more processors to perform a computer implemented process of detecting oil spill on a sea surface, the computer-implemented process including:
determining vertical emission rate, horizontal emission rate or reflectivity for each polarization for sea water and oil of a sea surface region on which oil is spilled, using radiance measured by a sensor mounted on a satellite, and
calculating the refractive index of the sea water and the oil using vertical reflectivity or horizontal reflectivity information determined at the determining step.

12. The method of claim 11, further comprising using the refractive indexes of the operated sea water and the oil to detect the region on which the oil is spilled.

13. The method of claim 12, wherein the determining obtains emission rate and vertical reflectivity or horizontal reflectivity for each polarization using the radiance and the sea surface temperature measured by the observation sensor unit including the infrared sensor of the satellite, where the reflectivity R(θ) and vertical reflectivity R_{V} or horizontal reflectivity R_{H} are calculated according to Equations 1 and 2 stated below,
$$R ( θ ) \approx 1 - \frac{{I}_{B} ( θ )}{B ( {T}_{S} )} \qquad \{ Equation 1 \}$$
where θ is an observation angle of the satellite
$${R}_{V} = {R}_{H}^{{sec}^{2} θ} \qquad \{ Equation 2 \}$$
where θ is the observation angle of the satellite and V and H represent vertical and horizontal polarizations, respectively.

14. The method of claim 13, wherein the calculating step processes the refractive indexes of the sea water and the oil band according to {Equation 3} using the reflectivity provided by the determining
$$n = \sqrt{\frac{{B}^{2} - {A}^{2} + {sin}^{2} θ + \sqrt{{( {A}^{2} + {B}^{2} - {sin}^{2} θ )}^{2} + 4 {A}^{2} {B}^{2}}}{2}} \qquad \{ Equation 3 \}$$
where A²=B²−2ab cos θ−cos²θ, B=(a−b)sin θ cot 2θ/[ab+(1−a²) cos²θ−1], coefficients a and b are a combination of reflectivities, wherein a=(R_{V}+1)/(R_{V}−1), and b=(R_{H}+1)/(R_{H}−1).

15. The method of claim 11, wherein the determining obtains emission rate and vertical reflectivity or horizontal reflectivity for each polarization using the radiance and the sea surface temperature measured by the observation sensor unit including the infrared sensor of the satellite, where the reflectivity R(θ) and vertical reflectivity R_{V} or horizontal reflectivity R_{H} are calculated according to Equations 1 and 2 stated below,
$$R ( θ ) \approx 1 - \frac{{I}_{B} ( θ )}{B ( {T}_{S} )} \qquad \{ Equation 1 \}$$
where θ is an observation angle of the satellite
$${R}_{V} = {R}_{H}^{{sec}^{2} θ} \qquad \{ Equation 2 \}$$
where θ is the observation angle of the satellite and V and H represent vertical and horizontal polarizations, respectively.

16. The method of claim 15, wherein the calculating step processes the refractive indexes of the sea water and the oil band according to {Equation 3} using the reflectivity provided by the determining step:
$$n = \sqrt{\frac{{B}^{2} - {A}^{2} + {sin}^{2} θ + \sqrt{{( {A}^{2} + {B}^{2} - {sin}^{2} θ )}^{2} + 4 {A}^{2} {B}^{2}}}{2}} \qquad \{ Equation 3 \}$$
where A²=B²−2ab cos θ−cos²θ, B=(a−b)sin θ cot 2θ/[ab+(1−a²) cos²θ−1], coefficients a and b are a combination of reflectivities, wherein a=(R_{V}+1)/(R_{V}−1), and b=(R_{H}+1)/(R_{H}−1).

17. A computer-readable medium including a program performing a system for detecting oil spills on the sea surface of claim 1 or a method for detecting oil spills on the sea surface claimed in claim 4.
